(19) United States
(12) Patent Application Publication
(10) Pub. No.: US 2025/0152048 A1
(43) Pub. Date: May 15, 2025

(54) HEALTH ANALYSIS METHOD AND APPARATUS, ELECTRONIC DEVICE AND STORAGE MEDIUM

(71) Applicant: BOE Technology Group Co., Ltd., Beijing (CN)

(72) Inventors: Congbin Li, Beijing (CN); Zhou Wang, Beijing (CN)

(73) Assignee: BOE Technology Group Co., Ltd., Beijing (CN)

(21) Appl. No.: 18/834,376

(22) PCT Filed: Apr. 27, 2023
(86) PCT No.: PCT/CN2023/091319

§ 371 Date: Jul. 30, 2024

(30) Foreign Application Priority Data
Apr. 29, 2022 (CN) 202210476067.7

(51) Int. Cl. A61B 5/145; A61B 5/00; A61B 5/0205; A61B 5/11; G16H 10/40; G16H 50/20

(52) U.S. Cl. CPC A61B 5/14532; A61B 5/0205; A61B 5/7275; G16H 10/40; G16H 50/20; A61B 5/11

(57) ABSTRACT

The present disclosure discloses to a health analysis method for blood glucose management, a health analysis apparatus for blood glucose management, an electronic device and a storage medium. The health analysis method includes: obtaining a plurality of blood glucose data of a user during a detection time period, wherein the plurality of blood glucose data include a current blood glucose value detected at a current moment; determining a time in range ratio of the user according to the plurality of blood glucose data and a glucose standard range; predicting a time when a blood glucose value of the user meets the glucose standard range and a time when the time in range ratio meets a qualified condition according to the current blood glucose value and the time in range ratio; and analyzing predicted results to provide a blood glucose prediction curve to the user.

### CROSS-REFERENCE TO RELATED APPLICATION

The present disclosure claims the priority of Chinese patent application filed on Apr. 29, 2022 before the CNIPA, China National Intellectual Property Administration with the application number of 202210476067.7, and the title of “HEALTH ANALYSIS METHOD AND APPARATUS, ELECTRONIC DEVICE AND STORAGE MEDIUM”, which is incorporated herein in its entirety by reference.

### FIELD

The present disclosure relates to the field of health, and more particularly to a health analysis method for blood glucose management, a health analysis apparatus for blood glucose management, an electronic device and a storage medium.

### BACKGROUND

In the related art, health assessments of patients given by blood glucose control applications can only provide general health management recommendations according to basic circumstances of the patients, and cannot provide guidance on blood glucose control for the patients in real time. It is necessary for the patients to constantly consult health managers/doctors, which brings many inconveniences to daily blood glucose control of the patients.

### SUMMARY

The present disclosure aims to solve at least one of technical problems in the related art. Therefore, the present disclosure provides a health analysis method for blood glucose management, a health analysis apparatus for blood glucose management, an electronic device and a storage medium.

The health analysis method for blood glucose management according to embodiments of the present disclosure includes:
obtaining a plurality of blood glucose data of a user during a detection time period, wherein the plurality of blood glucose data include a current blood glucose value detected at a current moment;
determining a time in range ratio of the user according to the plurality of blood glucose data and a glucose standard range;
predicting a time when a blood glucose value of the user meets the glucose standard range and a time when the time in range ratio meets a qualified condition according to the current blood glucose value and the time in range ratio; and analyzing predicted results to provide a blood glucose prediction curve to the user.

In some embodiments, the health analysis method further includes:
analyzing the predicted results to provide a blood glucose management path to the user.

In some embodiments, the health analysis method further includes:
analyzing the predicted results to provide a targeted target to the user, wherein the targeted target includes a predicted target blood glucose value and a time for reaching the predicted target blood glucose value.

In some embodiments, determining the time in range ratio of the user according to the plurality of blood glucose data and the glucose standard range includes:
determining a number of standard detections, wherein one or more blood glucose values in the blood glucose data obtained by performing the standard detections are within the glucose standard range; and
determining the time in range ratio according to a ratio of the number of standard detections to a total number of detections.

In some embodiments, predicting the time when the blood glucose value of the user meets the glucose standard range and the time when the time in range ratio meets the qualified condition according to the current blood glucose value and the time in range ratio includes:
in response to the current blood glucose value being higher than the glucose standard range, predicting a first predicted time when the blood glucose value of the user meets the glucose standard range according to the current blood glucose value and a blood glucose decreasing speed; and
predicting a second predicted time when the time in range ratio meets the qualified condition according to the first predicted time and the time in range ratio.

In some embodiments, the health analysis method includes:
obtaining motion data of the user in real time; and
determining the blood glucose decreasing speed according to the motion data.

In some embodiments, predicting the second predicted time when the time in range ratio meets the qualified condition according to the first predicted time and the time in range ratio includes:
determining a number of first predicted detections required to be experienced in that the blood glucose value of the user meets the glucose standard range according to the first predicted time;
determining a number of second predicted detections required to be experienced in predicting that the time in range ratio meets the qualified condition according to the number of first predicted detections, the number of standard detections and the total number of detections; and

determining the second predicted time according to the number of second predicted detections.

In some embodiments, analyzing the predicted results to provide the blood glucose prediction curve to the user includes:
determining a predicted target blood glucose value; and
drawing the blood glucose prediction curve according to the predicted target blood glucose value, the current blood glucose value, the first predicted time, the second predicted time and the blood glucose decreasing speed.

In some embodiments, determining the predicted target blood glucose value includes:
calculating a blood glucose estimated value according to the current blood glucose value, the first predicted time, the second predicted time and the blood glucose decreasing speed;
in response to the blood glucose estimated value being greater than or equal to a first preset value, determining the blood glucose estimated value as the predicted target blood glucose value; and
in response to the blood glucose estimated value being less than the first preset value, determining the first preset value as the predicted target blood glucose value.

In some embodiments, the targeted target is determined according to the predicted target blood glucose value, the first predicted time and the second predicted time.

In some embodiments, analyzing the predicted results to provide the blood glucose management path to the user includes:
determining a predicted motion time of the user according to the current blood glucose value, the blood glucose decreasing speed and the predicted target blood glucose value; and
providing the blood glucose management path according to the predicted motion time.

In some embodiments, providing the blood glucose management path according to the predicted motion time includes:
providing the blood glucose management path according to the predicted motion time and a current time; or
providing the blood glucose management path according to the predicted motion time and user personal data.

In some embodiments, predicting the time when the blood glucose value of the user meets the glucose standard range and the time when the time in range ratio meets the qualified condition according to the current blood glucose value and the time in range ratio includes:

in response to the current blood glucose value being within the glucose standard range, predicting a second predicted time when the time in range ratio meets the qualified condition according to the time in range ratio.

In some embodiments, predicting the time when the blood glucose value of the user meets the glucose standard range and the time when the time in range ratio meets the qualified condition according to the current blood glucose value and the time in range ratio includes:
in response to the current blood glucose value being lower than the glucose standard range, predicting a first predicted time when the blood glucose value of the user meets the glucose standard range according to the current blood glucose value and a blood glucose rising speed; and
predicting a second predicted time when the time in range ratio meets the qualified condition according to the first predicted time and the time in range ratio.

In some embodiments, predicting the second predicted time when the time in range ratio meets the qualified condition according to the first predicted time and the time in range ratio includes:
determining a number of first predicted detections required to be experienced in that the blood glucose value of the user meets the glucose standard range according to the first predicted time;
determining a number of second predicted detections required to be experienced in predicting that the time in range ratio meets the qualified condition according to the number of first predicted detections, the number of standard detections and the total number of detections; and
determining the second predicted time according to the number of second predicted detections.

In some embodiments, analyzing the predicted results to provide the blood glucose prediction curve to the user includes:
determining a predicted target blood glucose value; and
drawing the blood glucose prediction curve according to the predicted target blood glucose value, the current blood glucose value, the first predicted time, the second predicted time and the blood glucose rising speed.

In some embodiments, determining the predicted target blood glucose value includes: calculating a blood glucose estimated value according to the current blood glucose value,
the first predicted time, the second predicted time and the blood glucose rising speed;
in response to the blood glucose estimated value being less than or equal to a second preset value, determining the blood glucose estimated value as the predicted target blood glucose value; and
in response to the blood glucose estimated value being greater than the second preset value, determining the second preset value as the predicted target blood glucose value.

In some embodiments, when the time in range ratio meets the qualified condition includes: the time in range ratio is greater than or equal to 70%.

The health analysis apparatus for blood glucose management according to the embodiments of the present disclosure includes:
an obtaining module configured to obtain a plurality of blood glucose data of a user during a detection time period, wherein the plurality of blood glucose data include a current blood glucose value detected at a current moment;
a determination module configured to determine a time in range ratio of the user according to the plurality of blood glucose data and a glucose standard range;
a prediction module configured to predict a time when a blood glucose value of the user meets the glucose standard range and a time when the time in range ratio meets a qualified condition according to the current blood glucose value and the time in range ratio; and
an analysis module configured to analyze predicted results to provide a blood glucose prediction curve to the user.

The electronic device according to the embodiments of the present disclosure includes a processor and a memory, wherein the memory stores a computer program, and the computer program is executed by the processor to implement the health analysis method for blood glucose management.

A non-volatile computer-readable storage medium according to the embodiments of the present disclosure includes a computer program, wherein the computer program, when executed by one or more processors, causes the processors to implement the health analysis method for blood glucose management.

In the health analysis method for blood glucose management, the health analysis apparatus for blood glucose management, the electronic device and the computer storage medium according to the embodiments of the present disclosure, by calculating the time in range ratio of the user according to the blood glucose data collected from the user and the preset glucose standard range, predicting the time required for the blood glucose value of the user to meet the glucose standard range and the time required for the time in range ratio to meet the qualified condition according to the current blood glucose value and the time in range ratio, and analyzing the obtained predicted results to provide the blood glucose prediction curve to the user, the user can clearly see the gap and direction between the real-time health data of the user and the predicted standard blood glucose.

Additional aspects and advantages of the present disclosure will be partially given in the following description, and some will become apparent from the following description or will be learned through the practice of the present disclosure.

### BRIEF DESCRIPTION OF THE DRAWINGS

The above and/or additional aspects and advantages of the present disclosure will become apparent and easily understood from the following description of the embodiments taken in conjunction with the accompanying drawings, in which:

FIG. 1 is a flowchart illustrating a health analysis method for blood glucose management according to some embodiments of the present disclosure;

FIG. 2 is a schematic block diagram illustrating a health analysis apparatus for blood glucose management according to some embodiments of the present disclosure;

FIG. 3 is a schematic scenario diagram illustrating a health analysis method for blood glucose management according to some embodiments of the present disclosure; and

FIG. 4 to FIG. 18 are flowcharts illustrating a health analysis method for blood glucose management according to some embodiments of the present disclosure.

### DETAILED DESCRIPTION

Hereinafter, the embodiments of the present disclosure will be described in detail. Examples of the embodiments are illustrated in the accompanying drawings, where the same or similar reference numbers indicate the same or similar elements or elements with the same or similar functions throughout. The embodiments described below with reference to the accompanying drawings are exemplary and are intended to explain the present disclosure, but not to be construed as limitations of the present disclosure.

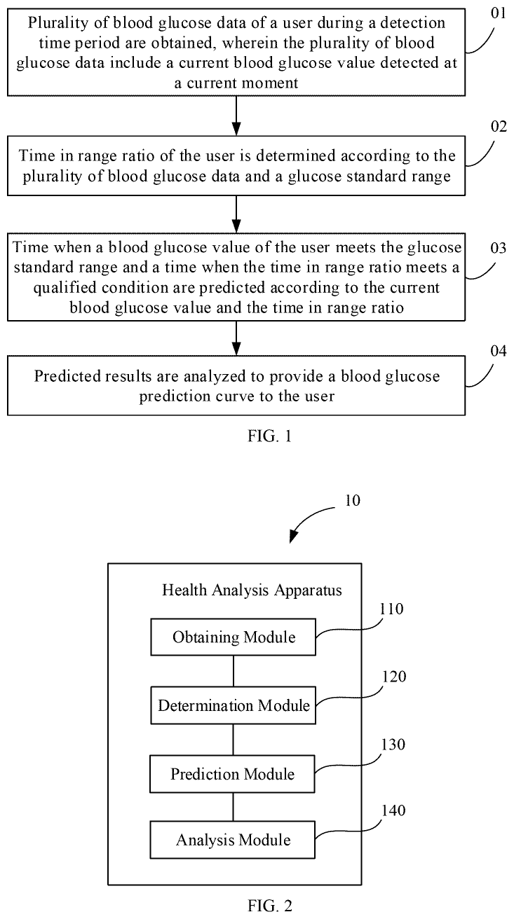

Referring to FIG. 1, the present disclosure provides a health analysis method for blood glucose management. The health analysis method includes the following steps:
01, a plurality of blood glucose data of a user during a detection time period are obtained, wherein the plurality of blood glucose data include a current blood glucose value detected at a current moment;
02, a time in range ratio of the user is determined according to the plurality of blood glucose data and a glucose standard range;
03, a time when a blood glucose value of the user meets the glucose standard range and a time when the time in range ratio meets a qualified condition are predicted according to the current blood glucose value and the time in range ratio; and
04, predicted results are analyzed to provide a blood glucose prediction curve to the user.

Referring to FIG. 2, the embodiment of the present disclosure provides a health analysis apparatus 10 for blood glucose management. The health analysis apparatus 10 includes an obtaining module 110, a determination module 120, a prediction module 130 and an analysis module 140. Step 01 can be implemented by the obtaining module 110, step 02 can be implemented by the determination module 120, and step 03 can be implemented by the prediction module 130. Step 04 can be implemented by the analysis module 140.

In other words, the obtaining module 110 can be configured to obtain a plurality of blood glucose data of a user during a detection time period, wherein the plurality of blood glucose data include a current blood glucose value detected at a current moment. The determination module 120 can be configured to determine a time in range ratio of the user according to the plurality of blood glucose data and a glucose standard range. The prediction module 130 can be configured to predict a time when a blood glucose value of the user meets the glucose standard range and a time when the time in range ratio meets a qualified condition according to the current blood glucose value and the time in range ratio. The analysis module 140 can be configured to analyze predicted results to provide a blood glucose prediction curve to the user.

The embodiment of the present disclosure further provides an electronic device. The electronic device includes a processor and a memory. The memory stores a computer program, and the computer program is executed by the processor, so that the processor can be configured to obtain a plurality of blood glucose data of a user during a detection time period, wherein the plurality of blood glucose data include a current blood glucose value detected at a current moment; determine a time in range ratio of the user according to the plurality of blood glucose data and a glucose standard range; predict a time when a blood glucose value of the user meets the glucose standard range and a time when the time in range ratio meets a qualified condition according to the current blood glucose value and the time in range ratio; and analyze predicted results to provide a blood glucose prediction curve to the user.

In the health analysis method for blood glucose management, the health analysis apparatus 10 for blood glucose management and the electronic device according to the embodiments of the present disclosure, by calculating the time in range ratio of the user according to the blood glucose data collected from the user and the preset glucose standard range, predicting the time required for the blood glucose value of the user to meet the glucose standard range and the time required for the time in range ratio to meet the qualified condition according to the current blood glucose value and the time in range ratio, and analyzing the obtained predicted results to provide the blood glucose prediction curve to the user, the user can clearly see the gap and direction between the real-time health data of the user and the predicted standard blood glucose.

In some embodiments, the health analysis apparatus 10 may be a part of the electronic device. In other words, the electronic device includes the health analysis apparatus 10.

In some embodiments, the health analysis apparatus 10 may be a discrete component assembled in a certain manner to have the aforementioned functions, or a chip with the aforementioned functions exists in the form of an integrated circuit, or a computer software code segment that enables a computer to have the aforementioned functions when running on the computer.

In some embodiments, as hardware, the health analysis apparatus 10 may be independent or added to an electronic device as an additional peripheral component. The health analysis apparatus 10 may also be integrated into an electronic device. For example, when the health analysis apparatus 10 is a part of an electronic device, the health analysis apparatus 10 may be integrated into a processor.

The electronic device can include but not limited to smart blood glucose devices (for example, smart blood glucose meters), mobile phones, computers, smart wearable devices (smartwatches, smart bracelets, smart helmets, smart glasses, or the like), virtual reality devices or head-mounted display devices.

In the embodiment, taking the electronic device as a smart blood glucose device as an example, that is, the health analysis method and the health analysis apparatus 10 can be applied to, but not limited to, the smart blood glucose device. The health analysis apparatus 10 may be hardware or software pre-installed on the smart blood glucose device, and may perform the health analysis method when the smart blood glucose device is started. For example, the health analysis method can be an underlying software code segment of the smart blood glucose device or a part of an operating system. In this way, when the blood glucose data of the user is obtained, the smart blood glucose device can provide the blood glucose prediction curve to the user, thereby enabling the user to realize blood glucose control.

In the embodiment, a detection frequency during the detection time period can be 6 times per hour (the blood glucose data of the user is detected once every 10 minutes).

It should be noted that the current blood glucose value detected at the current moment refers to a latest blood glucose value detected at the current moment. It can be understood that since the blood glucose value is measured every ten minutes, a blood glucose value detected last time can be used as the current blood glucose value when the current moment is not a detection time.

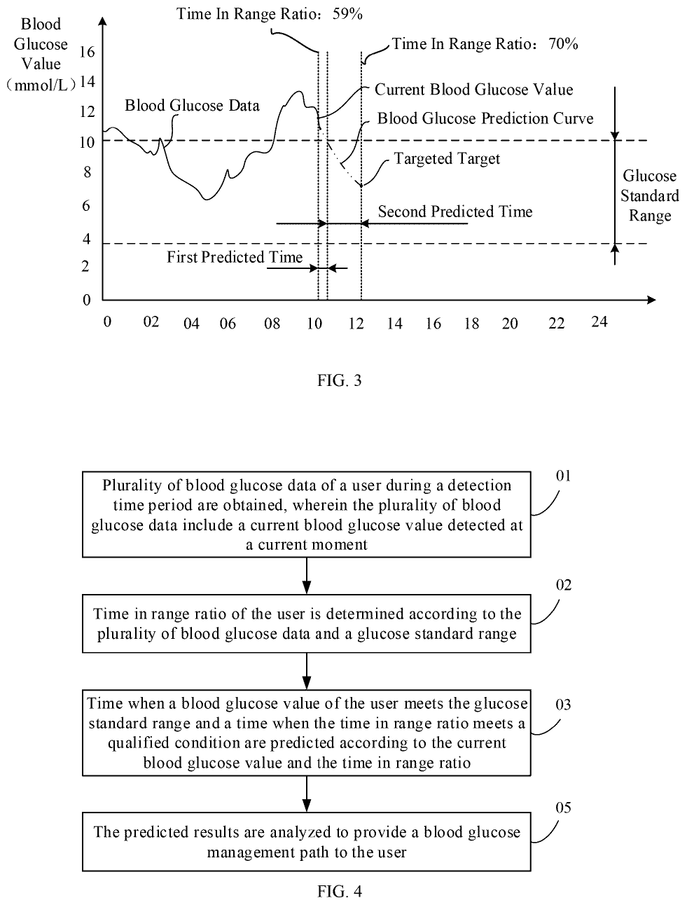

It should be noted that the time in range (TIR) ratio refers to a percentage of glucose within a target range (usually 3.9˜10.0 mmol/L, or 3.9˜7.8 mmol/L) within 24 hours, which is currently used as a new blood glucose control index. Referring to FIG. 3, in the present disclosure, a glucose target range can be 3.9˜10.0 mmol/L, that is, the glucose standard range in the present disclosure is 3.9˜10.0 mmol/L. The glucose standard range can be adjusted according to medical and practical needs. The present disclosure is only for illustrative purposes and does not limit the scope of the present disclosure.

The time in range ratio meets the qualified condition can be that the time in range ratio is not less than a predetermined threshold, which can be 65%, 70%, 75%, 80%, 85%, or 90%, and the specific size of the predetermined threshold is not limited. For example, in the embodiment, the time in range ratio meets the qualified condition can be that the time in range ratio is not less than 70%, that is, if the blood glucose value is within the glucose target range for a period of time greater than or equal to 70% during the detection time period, it can be considered that the blood glucose of the user is qualified.

The time when the blood glucose value meets the glucose standard range refers to a time required to adjust the blood glucose value to the glucose standard range. For example, the current blood glucose value is 14 mmol/L, and the glucose standard range is 3.9˜10.0 mmol/L, which means that the time required for the blood glucose value to drop from 14 mmol/L to 3.9˜10.0 mmol/L. It should be noted that no matter whether the blood glucose value is higher than the glucose standard range or lower than the glucose standard range, it can be considered that the blood glucose value has not reached the standard, and it is necessary to reduce or increase the blood glucose value, so that the blood glucose value can meet the glucose standard range.

The blood glucose prediction curve is used to predict a general trend of subsequent blood glucose values of the user.

In some embodiments, the blood glucose prediction curve can be directly predicted based on data (for example, diets, medicines, motions, and the like) marked by the user.

Referring to FIG. 4, in some embodiments, the health analysis method further includes:
05, the predicted results are analyzed to provide a blood glucose management path to the user.

In some embodiments, step 05 can be implemented by the analysis module 140, in other words, the analysis module 140 can be configured to analyze the predicted results to provide a blood glucose management path to the user.

In some embodiments, the processor can be configured to analyze the predicted results to provide a blood glucose management path to the user.

It should be noted that the blood glucose management path refers to providing suggestions to the user to meet blood glucose control standards. The blood glucose management path can include but not limited to suggestions on diets, motions and medicines.

In some embodiments, the blood glucose prediction curve can be generated based on the blood glucose management path. The blood glucose management path can include one or more paths. When there are multiple blood glucose management paths, each of the multiple blood glucose management paths generates a corresponding blood glucose prediction curve. In this way, it is convenient for the user to choose the management path suitable for him/her.

In addition, in some embodiments, actual blood glucose data obtained by the user according to the provided blood glucose management path can be recorded, and an actual blood glucose curve can be generated according to the actual blood glucose data. Further, a blood glucose control report can be generated by comparing the actual blood glucose curve with simulated data that is assumed not to be adopted by the user, and the blood glucose control report is fed back to the user.

Referring to FIG. 5, in some embodiments, the health analysis method further includes:
06, the predicted results are analyzed to provide a targeted target to the user, wherein the targeted target includes a predicted target blood glucose value and a time for reaching the predicted target blood glucose value.

In some embodiments, step 06 can be implemented by the analysis module 140, in other words, the analysis module 140 can be configured to analyze the predicted results to provide a targeted target to the user, wherein the targeted target includes a predicted target blood glucose value and a time for reaching the predicted target blood glucose value.

In some embodiments, the processor can be configured to analyze the predicted results to provide a targeted target to the user, wherein the targeted target includes a predicted target blood glucose value and a time for reaching the predicted target blood glucose value.

It should be noted that the targeted target can clearly show how long it will take the user to reach the target and what the target blood glucose value is. The closer the current moment is to the targeted target, the easier it is for the user to achieve the time in range ratio to meet the qualified condition. Conversely, the farther the current moment is to the targeted target, the more difficult it is for the user to achieve the time in range ratio to meet the qualified condition.

In this way, the user can intuitively know when the blood glucose can reach the standard and the blood glucose value when the blood glucose reaches the standard according to the targeted target.

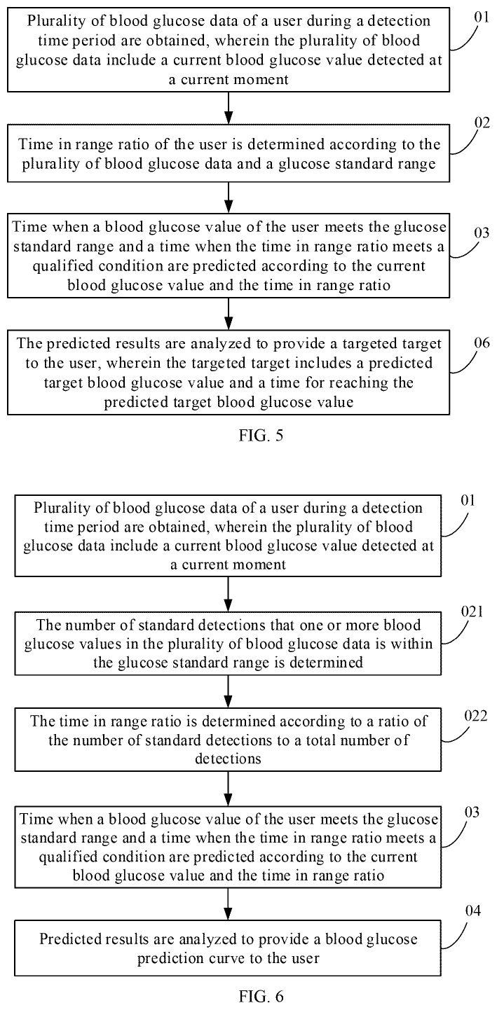

Referring to FIG. 6, in some embodiments, step 02 includes the following sub-steps:
021, a number of standard detections is determined, wherein one or more blood glucose values in the blood glucose data obtained by performing the standard detections are within the glucose standard range; and
022, the time in range ratio is determined according to a ratio of the number of standard detections to a total number of detections.

In some embodiments, sub-steps 021 and 022 can be implemented by the determination module 120, in other words, the determination module 120 can be configured to determine a number of standard detections, wherein one or more blood glucose values in the blood glucose data obtained by performing the standard detections are within the glucose standard range; and determine the time in range ratio according to a ratio of the number of standard detections to a total number of detections.

In some embodiments, the processor can be configured to determine a number of standard detections, wherein one or more blood glucose values in the blood glucose data obtained by performing the standard detections are within the glucose standard range; and determine the time in range ratio according to a ratio of the number of standard detections to a total number of detections.

For example, during the detection time period, the number of measured blood glucose values that reach the standard is a, the number of measured blood glucose values that fail to reach the standard is b, and the total number of measured blood glucose values is c, then the time in range ratio is a/c. It can be understood that when the total number of measured blood glucose values is constant, the greater the number of measured blood glucose values that reach the standard, the higher the time in range ratio, and the better the blood glucose status of the user.

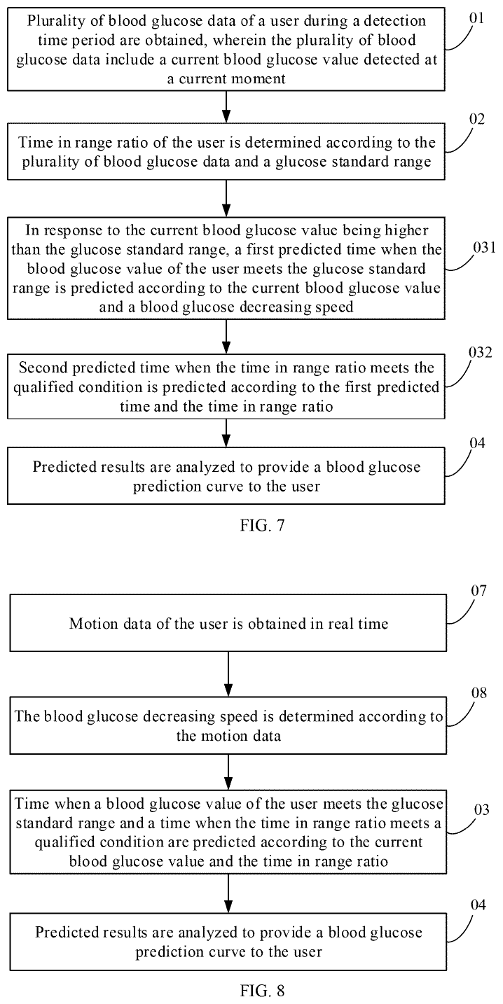

Referring to FIG. 7, in some embodiments, step 03 includes the following sub-steps:
031, in response to the current blood glucose value being higher than the glucose standard range, a first predicted time when the blood glucose value of the user meets the glucose standard range is predicted according to the current blood glucose value and a blood glucose decreasing speed; and
032, a second predicted time when the time in range ratio meets the qualified condition is predicted according to the first predicted time and the time in range ratio.

In some embodiments, sub-steps 031 and 032 can be implemented by the prediction module 130, in other words, the prediction module 130 can be configured to in response to the current blood glucose value being higher than the glucose standard range, predict a first predicted time when the blood glucose value of the user meets the glucose standard range according to the current blood glucose value and a blood glucose decreasing speed; and predict a second predicted time when the time in range ratio meets the qualified condition according to the first predicted time and the time in range ratio.

In some embodiments, the processor can be configured to in response to the current blood glucose value being higher than the glucose standard range, predict a first predicted time when the blood glucose value of the user meets the glucose standard range according to the current blood glucose value and a blood glucose decreasing speed; and predict a second predicted time when the time in range ratio meets the qualified condition according to the first predicted time and the time in range ratio.

That is, the first predicted time is the time when the blood glucose value of the user meets the glucose standard range. For example, the current blood glucose value is 10.2, and the time required for the blood glucose value to drop from 10.2 mmol/L to 3.9˜10.0 mmol/L is 0.2 hours, then the first predicted time is 0.2 hours. The second predicted time is the time when the time in range ratio meets the qualified condition.

In this way, the user can determine when his/her the blood glucose value can reach the standard according to the first predicted time, and can determine when his/her TIR is qualified according to the second predicted time.

Referring to FIG. 8, in some embodiments, before step 03, the health analysis method further includes:
07, motion data of the user is obtained in real time; and
08, the blood glucose decreasing speed is determined according to the motion data.

Further referring to FIG. 2, in some embodiments, step 07 can be implemented by the obtaining module 110 and step 08 can be implemented by the determination module 120. In other words, the obtaining module 110 can be configured to obtain motion data of the user in real time; and the determination module 120 can be configured to determine the blood glucose decreasing speed according to the motion data.

In some embodiments, the processor can be configured to obtain motion data of the user in real time; and determine the blood glucose decreasing speed according to the motion data.

In the embodiment, the motion data of the user can be obtained through smart Internet of Things (IoT) devices such as sports wristbands or sports watches, or the blood glucose prediction curve can be obtained by inputting a motion mode and a motion time into an electronic device before user motion.

It can be understood that different motion modes consume different blood glucose concentrations within a predetermined time. For example, in general, walking for one hour reduces blood glucose by 2 mmol/L, and jogging for one hour reduces blood glucose by 4 mmol/L. In the present disclosure, taking the user walking as an example, that is, the blood glucose decreasing speed is 2 mmol/L/hour. For example, if the current blood glucose value of the user is 12 mmol/L, and the user exercises by walking, the time required for the blood glucose value of the user to meet the glucose standard range is (12−10)/2, that is, 1 hour. In other ways, various motion modes (for example, jogging and walking) can be used to reduce the blood glucose value, and when the motion mode of the user changes interactively, the blood glucose prediction curve for each segment can be adjusted in time according to the motion mode and the motion time of each segment.

In this way, one or more suitable motion modes can be recommended to reduce the blood glucose data according to the current blood glucose data of the user.

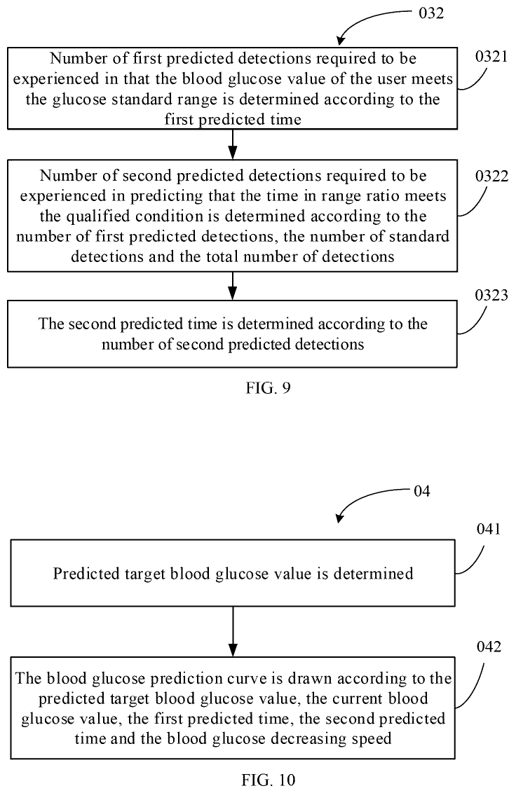

Referring to FIG. 9, in some embodiments, step 032 includes the following sub-steps:
0321, a number of first predicted detections required to be experienced in that the blood glucose value of the user meets the glucose standard range is determined according to the first predicted time;
0322, a number of second predicted detections required to be experienced in predicting that the time in range ratio meets the qualified condition is determined according to the number of first predicted detections, the number of standard detections and the total number of detections; and
0323, the second predicted time is determined according to the number of second predicted detections.

In some embodiments, sub-steps 0321-0323 can be implemented by the prediction module 130, in other words, the prediction module 130 can be configured to determine a number of first predicted detections required to be experienced in that the blood glucose value of the user meets the glucose standard range according to the first predicted time; determine a number of second predicted detections required to be experienced in predicting that the time in range ratio meets the qualified condition according to the number of first predicted detections, the number of standard detections and the total number of detections; and determine the second predicted time according to the number of second predicted detections.

In some embodiments, the processor can be configured to determine a number of first predicted detections required to be experienced in that the blood glucose value of the user meets the glucose standard range according to the first predicted time; determine a number of second predicted detections required to be experienced in predicting that the time in range ratio meets the qualified condition according to the number of first predicted detections, the number of standard detections and the total number of detections; and determine the second predicted time according to the number of second predicted detections.

It can be understood that in the present disclosure, the blood glucose data is collected 6 times per hour, it is predicted that the time in range ratio is the ratio of the number of standard detections to the total number of detections, and the time in range ratio needs to be not less than 70%. Further referring to FIG. 3, in some examples, the current blood glucose value of the user is 10.6 mmol/L, the total number of detections during the current detection time period is 54, and the number of standard detections that the blood glucose value meets the glucose standard range is 32, then the time in range ratio of the current user is 32/54=0.59, at this time, the time in range ratio of the user does not meet the qualified condition. If the user walks to reduce blood glucose, the first predicted time for the blood glucose of the user to meet the standard range is (10.6−10)/2=0.3 hours, that is, the time required for the blood glucose value of the user to meet the standard range is 0.3 hours and 18 minutes, and thus the number of first predicted detections required to be experienced is 1.8 times, which is about 2 times.

Further, let the number of second predicted detections be x, since it is predicted that the time in range ratio is the ratio of the number of standard detections to the total number of detections, and the ratio is greater than or equal to 70%, it can be obtained that (32+x)/(54+2+x) is not less than 70%, the number of second predicted detections are calculated to be x=24, and the second predicted time is 24/6=4 hours. That is, in the current situation, if the user walks, the blood glucose value can meet the glucose standard range in about 0.3 hours, and it is estimated that the state within the glucose standard range needs to be maintained for 4 hours to make the time in range ratio meet the qualified condition.

Referring to FIG. 10, in some embodiments, step 04 includes the following sub-steps:
041, a predicted target blood glucose value is determined; and
042, the blood glucose prediction curve is drawn according to the predicted target blood glucose value, the current blood glucose value, the first predicted time, the second predicted time and the blood glucose decreasing speed.

In some embodiments, sub-steps 041 and 042 can be implemented by the analysis module 140, in other words, the analysis module 140 can be configured to determine a predicted target blood glucose value; and draw the blood glucose prediction curve according to the predicted target blood glucose value, the current blood glucose value, the first predicted time, the second predicted time and the blood glucose decreasing speed.

In some embodiments, the processor can be configured to determine a predicted target blood glucose value; and draw the blood glucose prediction curve according to the predicted target blood glucose value, the current blood glucose value, the first predicted time, the second predicted time and the blood glucose decreasing speed.

In the embodiment, considering that diabetic patients need to pay attention to the postprandial 2-hour period and postprandial blood glucose, the predicted target blood glucose value can be a value between 4.4 mmol/L and 7.8 mmol/L. For example, when the current blood glucose value of the user is higher than the glucose standard range, the predicted target blood glucose value can be 4.4 mmol/L.

The predicted target blood glucose value is a blood glucose value of the user at a target moment, and the predicted target blood glucose value is a blood glucose value at the end of the blood glucose prediction curve. The current blood glucose value is a blood glucose value at the beginning of the blood glucose prediction curve. The blood glucose decreasing speed is a curvature of the blood glucose prediction curve. The second predicted time is a time period at the end of the blood glucose prediction curve.

In this way, the user can intuitively understand the changes of his/her own blood glucose value based on the blood glucose management path according to the blood glucose prediction curve.

In addition, in some embodiments, the electronic device is also preset with a blood glucose change setting value, which can be a standard value. The blood glucose prediction curve can also be drawn according to the predicted target blood glucose value, the current blood glucose value, the first predicted time, the second predicted time and blood glucose change standard parameters.

In some embodiments, the blood glucose prediction curve can also be drawn according to the predicted target blood glucose value, the current blood glucose value, the first predicted time, the second predicted time and parameters trained by deep learning based on the personal data of the user. In this way, the blood glucose prediction curve can most accurately conform to actual blood glucose changes of the user.

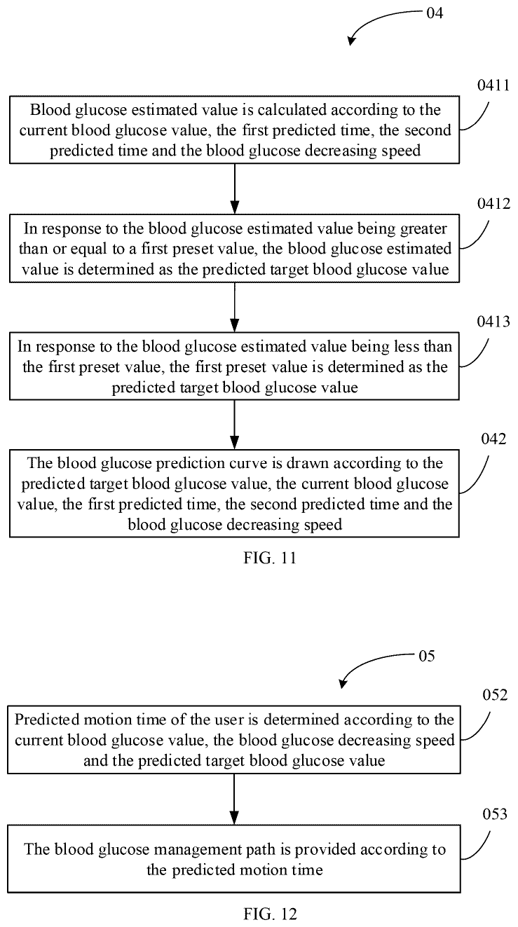

Referring to FIG. 11, in some embodiments, step 041 includes the following sub-steps:
0411, a blood glucose estimated value is calculated according to the current blood glucose value, the first predicted time, the second predicted time and the blood glucose decreasing speed;
0412, in response to the blood glucose estimated value being greater than or equal to a first preset value, the blood glucose estimated value is determined as the predicted target blood glucose value; and
0413, in response to the blood glucose estimated value being less than the first preset value, the first preset value is determined as the predicted target blood glucose value.

In some embodiments, sub-steps 0411-0413 can be implemented by the analysis module 140, in other words, the analysis module 140 can be configured to calculate a blood glucose estimated value according to the current blood glucose value, the first predicted time, the second predicted time and the blood glucose decreasing speed; in response to the blood glucose estimated value being greater than or equal to a first preset value, determine the blood glucose estimated value as the predicted target blood glucose value; and in response to the blood glucose estimated value being less than the first preset value, determine the first preset value as the predicted target blood glucose value.

In some embodiments, the processor can be configured to calculate a blood glucose estimated value according to the current blood glucose value, the first predicted time, the second predicted time and the blood glucose decreasing speed; in response to the blood glucose estimated value being greater than or equal to a first preset value, determine the blood glucose estimated value as the predicted target blood glucose value; and in response to the blood glucose estimated value being less than the first preset value, determine the first preset value as the predicted target blood glucose value.

The blood glucose estimated value refers to a blood glucose value of the user after the first predicted time and the second predicted time. The first preset value can be 4.4 mmol/L, that is, when the blood glucose estimated value is not less than 4.4 mmol/L, it is determined that the blood glucose estimated value is the predicted target blood glucose value. When the blood glucose estimated value is less than 4.4 mmol/L, 4.4 mmol/L is taken as the predicted target blood glucose value. When the user exercises according to the blood glucose management path, if the blood glucose value drops to the predicted target blood glucose value, the exercise is stopped.

It can be understood that with the increase of exercise duration, the blood glucose value will decrease. If the first preset time and the second preset time are too long, the blood glucose value of the user may be lower than the glucose standard range, resulting in hypoglycemia.

For example, in some examples, the user is in a hyperglycemic state at the current moment, and the blood glucose value is 10.6 mmol/L. The user uses walking exercise to reduce the blood glucose value, the first predicted time is 0.3 hours, and the second predicted time is 4 hours. That is, the user can exercise for 0.3 hours at the current moment, so that the blood glucose value can be reduced to the glucose standard range. However, if the exercise is stopped, it is likely that the blood glucose value will fluctuate, and the blood glucose value will not be within the glucose standard range in the subsequent time, resulting in that the time in range ratio after the first predicted time and the second predicted time cannot meet the qualified condition. If the user continues to walk for the next 4.3 hours, it can be concluded that the blood glucose value of the user will be 10.6−(2*4.3)=2 mmol/L after the first predicted time and the second predicted time, and the blood glucose value of 2 mmol/L is a serious hypoglycemia state.

In this way, by determining the blood glucose estimated value as the predicted target blood glucose value when the blood glucose estimated value is not less than the first preset value, and determining the first preset value as the predicted target blood glucose value when the blood glucose estimated value is less than the first preset value, it is possible to ensure that the blood glucose value of the user at the second predicted time is within the standard range while the time in range ratio meets the qualified condition after the first predicted time and the second predicted time.

In some embodiments, step 05 includes a sub-step:
051, the targeted target is determined according to the predicted target blood glucose value, the first predicted time and the second predicted time.

In some embodiments, sub-step 051 can be implemented by the analysis module 140, in other words, the analysis module 140 can be configured to determine the targeted target according to the predicted target blood glucose value, the first predicted time and the second predicted time.

In some embodiments, the processor can be configured to determine the targeted target according to the predicted target blood glucose value, the first predicted time and the second predicted time.

In this way, the user can intuitively know a duration required for the time in range ratio to meet the qualified condition and the blood glucose value at the duration according to the targeted target.

Referring to FIG. 12, in some embodiments, step 05 further includes the following sub-steps:
052, a predicted motion time of the user is determined according to the current blood glucose value, the blood glucose decreasing speed and the predicted target blood glucose value; and
053, the blood glucose management path is provided according to the predicted motion time.

In some embodiments, sub-steps 052 and 053 can be implemented by the analysis module 140, in other words, the analysis module 140 can be configured to determine a predicted motion time of the user according to the current blood glucose value, the blood glucose decreasing speed and the predicted target blood glucose value; and the blood glucose management path according to the predicted motion time.

In some embodiments, the processor can be configured to determine a predicted motion time of the user according to the current blood glucose value, the blood glucose decreasing speed and the predicted target blood glucose value; and the blood glucose management path according to the predicted motion time.

For example, in some examples, the current blood glucose value of the user is 10.6 mmol/L, the predicted target blood glucose value is 4.4 mmol/L, and a walking exercise mode is adopted (the blood glucose decreasing speed is 2 mmol/L), then the predicted motion time of the user is (10.6−4.4)/2=3.1 hours. Therefore, it is suggested that the user can walk for 3.1 hours and control the blood glucose within the standard range according to the blood glucose management path.

For another example, in some examples, the current blood glucose value of the user is 10 mmol/L, the blood glucose decreasing speed is 2 mmol/L, and the predicted target blood glucose value is 7 mmol/L, then the predicted motion time of the user is (10−7)/2=1.5 hours. Therefore, it is suggested that the user can walk for 1.5 hours and control the blood glucose within the standard range according to the blood glucose management path.

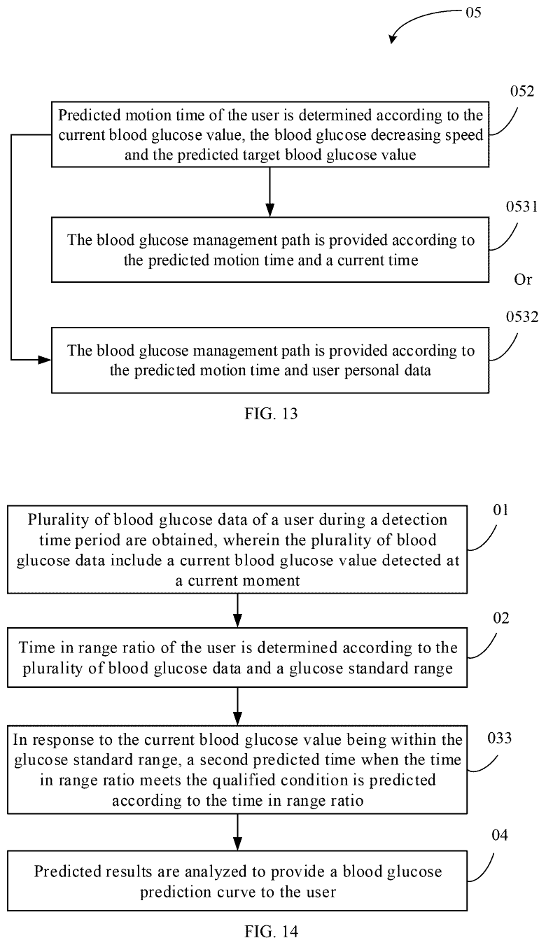

Referring to FIG. 13, in some embodiments, sub-step 053 includes:
0531, the blood glucose management path is provided according to the predicted motion time and a current time; or
0532, the blood glucose management path is provided according to the predicted motion time and user personal data.

In some embodiments, sub-steps 0531 and 0532 can be implemented by the analysis module 140, in other words, the analysis module 140 can be configured to provide the blood glucose management path according to the predicted motion time and a current time; or provide the blood glucose management path according to the predicted motion time and user personal data.

In some embodiments, the processor can be configured to provide the blood glucose management path according to the predicted motion time and a current time; or provide the blood glucose management path according to the predicted motion time and user personal data.

It should be noted that user personal data can be user physiological data or data generated according to user habits.

It can be understood that it may not be suitable for exercise or strenuous exercise during certain time periods, for example, at 2 μm, the user may be in working hours, and thus the blood glucose management path may not be suitable for providing exercise options. For example, at 9 μm, the user may not be suitable for strenuous exercise, and thus the blood glucose management path may not be suitable for providing running options. Therefore, by combining the predicted motion time, current time and user personal data to provide a blood glucose management path, the blood glucose management path can best fit the habits of the user, thereby improving user experience.

Referring to FIG. 14, in some embodiments, step 03 includes a sub-step:
033, in response to the current blood glucose value being within the glucose standard range, a second predicted time when the time in range ratio meets the qualified condition is predicted according to the time in range ratio.

In some embodiments, sub-step 033 can be implemented by the prediction module 130, in other words, the prediction module 130 can be configured to in response to the current blood glucose value being within the glucose standard range, predict a second predicted time when the time in range ratio meets the qualified condition according to the time in range ratio.

In some embodiments, the processor can be configured to in response to the current blood glucose value being within the glucose standard range, predict a second predicted time when the time in range ratio meets the qualified condition according to the time in range ratio.

For example, the current time is 10:00:00, during the detection time period from 0:00:00 to 10:00:00 and the current blood glucose value of the user is within the glucose standard range, the total number of detections is 60, and the number of blood glucose standard detections is 36, then the time in range ratio of the user is 36/60=0.6, which is less than 0.7, and thus the time in range ratio of the user does not meet the qualified condition. In order to make the time in range ratio of the user meet the qualified condition, let the number of blood glucose standard detections within the second predicted time is x, and the time in range ratio (36+x)/(60+x) is not less than 0.7, then x=20. Since the detection frequency of blood glucose data is 6 times per hour, the second predicted time is 20/6=3.33 hours.

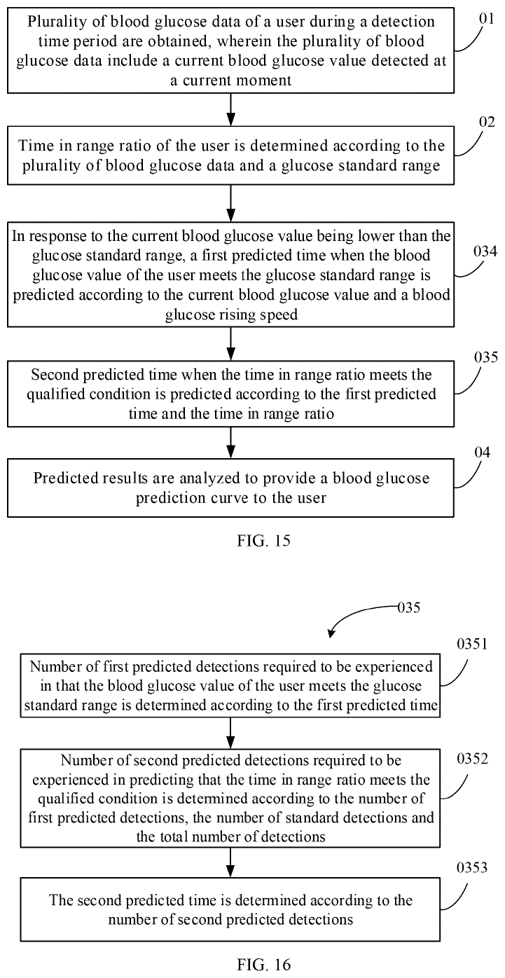

Referring to FIG. 15, in some embodiments, step 03 includes the following sub-steps:
034, in response to the current blood glucose value being lower than the glucose standard range, a first predicted time when the blood glucose value of the user meets the glucose standard range is predicted according to the current blood glucose value and a blood glucose rising speed; and
035, a second predicted time when the time in range ratio meets the qualified condition is predicted according to the first predicted time and the time in range ratio.

In some embodiments, sub-steps 034 and 035 can be implemented by the prediction module 130, in other words, the prediction module 130 can be configured to in response to the current blood glucose value being lower than the glucose standard range, predict a first predicted time when the blood glucose value of the user meets the glucose standard range according to the current blood glucose value and a blood glucose rising speed; and predict a second predicted time when the time in range ratio meets the qualified condition according to the first predicted time and the time in range ratio.

In some embodiments, the processor can be configured to in response to the current blood glucose value being lower than the glucose standard range, predict a first predicted time when the blood glucose value of the user meets the glucose standard range according to the current blood glucose value and a blood glucose rising speed; and predict a second predicted time when the time in range ratio meets the qualified condition according to the first predicted time and the time in range ratio.

It should be noted that in general, the blood glucose value can be increased by supplementing human energy through some methods such as eating, for example, eating at dinner time, adding meals when hypoglycemia occurs, or the like, and the range of blood glucose increase is affected by eating situation of the patient. The rising speed of blood glucose is positively correlated with a glycemic index (GI) of food.

The value of the first predicted time can be in the range of 0.5˜1 hour.

The glycemic index refers to the relative ability of different foods to raise the blood glucose rapidly when containing the same amount of sugar. The higher the glycemic index, the faster the blood glucose rising speed; and the lower the glycemic index, the slower the blood glucose rising speed. In general, the GI value of glucose is set to 100. For low GI foods, GI<55; for medium GI foods, GI=55˜70; and for high GI foods, GI>70.

In general, the formula for calculating postprandial blood glucose is as follows:

Postprandial blood glucose (mmol/L)=1.5*glycosylated hemoglobin (%)+0.5*fasting blood glucose (mmol/L)−4.1.

For example, if the fasting blood glucose is 3.0 mmol/L, and the glycosylated hemoglobin is 9.0%, then according to the above formula, the postprandial blood glucose of the user is 1.5*9+0.5*3−4.1=10.9 mmol/L.

In this way, the user can determine when his/her blood glucose value can reach the standard according to the first predicted time, and can determine when his/her TIR is qualified according to the second predicted time.

Referring to FIG. 16, in some embodiments, the step 035 includes the following sub-steps:
0351, a number of first predicted detections required to be experienced in that the blood glucose value of the user meets the glucose standard range is determined according to the first predicted time;
0352, a number of second predicted detections required to be experienced in predicting that the time in range ratio meets the qualified condition is determined according to the number of first predicted detections, the number of standard detections and the total number of detections; and
0353, the second predicted time is determined according to the number of second predicted detections.

In some embodiments, sub-steps 0351 and 0352 can be implemented by the prediction module 130, in other words, the prediction module 130 can be configured to determine a number of first predicted detections required to be experienced in that the blood glucose value of the user meets the glucose standard range according to the first predicted time; determine a number of second predicted detections required to be experienced in predicting that the time in range ratio meets the qualified condition according to the number of first predicted detections, the number of standard detections and the total number of detections; and determine the second predicted time according to the number of second predicted detections.

In some embodiments, the processor can be configured to determine a number of first predicted detections required to be experienced in that the blood glucose value of the user meets the glucose standard range according to the first predicted time; determine a number of second predicted detections required to be experienced in predicting that the time in range ratio meets the qualified condition according to the number of first predicted detections, the number of standard detections and the total number of detections; and determine the second predicted time according to the number of second predicted detections.

It can be understood that in the present disclosure, the blood glucose data is collected 6 times per hour, it is predicted that the time in range ratio is the ratio of the number of standard detections to the total number of detections, and the time in range ratio needs to be not less than 70%. In some examples, if the first predicted time is 2 hours, the number of first predicted detections is 12, the number of standard detections is 28, the total number of detections is 40, and the second predicted time is 280 minutes.

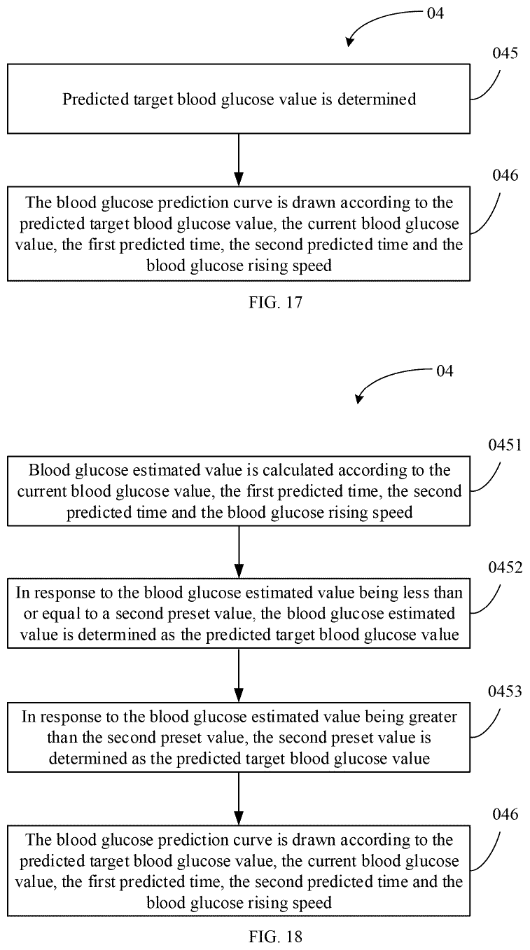

Referring to FIG. 17, in some embodiments, step 04 includes the following sub-steps:
045, a predicted target blood glucose value is determined; and

046, the blood glucose prediction curve is drawn according to the predicted target blood glucose value, the current blood glucose value, the first predicted time, the second predicted time and the blood glucose rising speed.

In some embodiments, sub-steps 045 and 046 can be implemented by the analysis module 140, in other words, the analysis module 140 can be configured to determine a predicted target blood glucose value; and draw the blood glucose prediction curve according to the predicted target blood glucose value, the current blood glucose value, the first predicted time, the second predicted time and the blood glucose rising speed.

In some embodiments, the processor can be configured to determine a predicted target blood glucose value; and draw the blood glucose prediction curve according to the predicted target blood glucose value, the current blood glucose value, the first predicted time, the second predicted time and the blood glucose rising speed.

The predicted target blood glucose value can be a value between 4.4 mmol/L and 7.8 mmol/L. For example, when the current blood glucose value of the user is lower than the glucose standard range, the predicted target blood glucose value can be 7.8 mmol/L.

The predicted target blood glucose value is a blood glucose value at the end of the blood glucose prediction curve. The current blood glucose value is a blood glucose value at the beginning of the blood glucose prediction curve. The blood glucose rising speed is a curvature of the blood glucose prediction curve. The second predicted time is a time period at the end of the blood glucose prediction curve.

In this way, the user can intuitively understand the changes of his/her own blood glucose value based on the blood glucose management path according to the blood glucose prediction curve.

Referring to FIG. 18, in some embodiments, step 045 includes the following sub-steps:
0451, a blood glucose estimated value is calculated according to the current blood glucose value, the first predicted time, the second predicted time and the blood glucose rising speed;
0452, in response to the blood glucose estimated value being less than or equal to a second preset value, the blood glucose estimated value is determined as the predicted target blood glucose value; and
0453, in response to the blood glucose estimated value being greater than the second preset value, the second preset value is determined as the predicted target blood glucose value.

In some embodiments, sub-steps 0451-0453 can be implemented by the analysis module 140, in other words, the analysis module 140 can be configured to calculate a blood glucose estimated value according to the current blood glucose value, the first predicted time, the second predicted time and the blood glucose rising speed; in response to the blood glucose estimated value being less than or equal to a second preset value, determine the blood glucose estimated value as the predicted target blood glucose value; and in response to the blood glucose estimated value being greater than the second preset value, determine the second preset value as the predicted target blood glucose value.

In some embodiments, the processor can be configured to calculate a blood glucose estimated value according to the current blood glucose value, the first predicted time, the second predicted time and the blood glucose rising speed; in response to the blood glucose estimated value being less than or equal to a second preset value, determine the blood glucose estimated value as the predicted target blood glucose value; and in response to the blood glucose estimated value being greater than the second preset value, determine the second preset value as the predicted target blood glucose value.

The second preset value can be 7.8 mmol/L, that is, when the blood glucose estimated value is not less than 7.8 mmol/L, it is determined that the blood glucose estimated value is the predicted target blood glucose value. When the blood glucose estimated value is less than 7.8 mmol/L, 7.8 mmol/L is taken as the predicted target blood glucose value. When the user exercises according to the blood glucose management path, if the blood glucose value rises to the predicted target blood glucose value, the exercise is stopped.

In this way, by determining the blood glucose estimated value as the predicted target blood glucose value when the blood glucose estimated value is not greater than the second preset value, and determining the second preset value as the predicted target blood glucose value when the blood glucose estimated value is greater than the second preset value, it is possible to ensure that the blood glucose value of the user at the second predicted time is within the standard range and that the time in range ratio meets the qualified condition after the first predicted time and the second predicted time.

The embodiment of the present disclosure further provides a non-volatile computer-readable storage medium. The readable storage medium stores a computer program, wherein the computer program, when executed by one or more processors, causes the processors to implement the above health analysis method for blood glucose management.

All or some of the foregoing embodiments may be implemented by using software, hardware, firmware, or any combination thereof. When the software is used to implement the embodiments, the embodiments may be implemented entirely or partially in a form of a computer program product. The computer program product includes one or more computer instructions. When the computer program instructions are loaded and executed on a computer, the procedures or functions according to the embodiments of the present disclosure are all or partially generated. The computer may be a general-purpose computer, a special-purpose computer, a computer network, or another programmable apparatus. The computer instructions may be stored in a computer-readable storage medium or may be transmitted from a computer-readable storage medium to another computer-readable storage medium. For example, the computer instructions may be transmitted from a website, computer, server, or data center to another website, computer, server, or data center in a wired (for example, a coaxial cable, an optical fiber, or a digital subscriber line (DSL)) or wireless (for example, infrared, radio, or microwave) manner. The computer-readable storage medium may be any usable medium accessible by a computer, or a data storage device, for example, a server or a data center, integrating one or more usable media. The usable medium may be a magnetic medium (for example, a floppy disk, a hard disk, or a magnetic tape), an optical medium (for example, a digital video disc (DVD)), a semiconductor medium (for example, a solid-state drive (SSD)), or the like.

A person of ordinary skill in the art may be aware that the units and the algorithm steps in the examples described with reference to the embodiments disclosed in this specification may be implemented by electronic hardware or a combination of computer software and electronic hardware. Whether the functions are performed by hardware or software depends on particular applications and design constraints of the technical solutions. A person skilled in the art may use different methods to implement the described functions of each particular application, but it should not be considered that the implementation goes beyond the scope of the present disclosure.

In the several embodiments provided in the present disclosure, it should be understood that the disclosed system, apparatus, and method may be implemented in other manners. For example, the described apparatus embodiment is merely an example. For example, division into units is merely logical function division and may be other division in actual implementation. For example, a plurality of units or components may be combined or integrated into another system, or some features may be ignored or not performed. In addition, the displayed or discussed mutual couplings or direct couplings or communication connections may be implemented through some interfaces. The indirect couplings or communication connections between the apparatuses or units may be implemented in electronic, mechanical, or other forms.

In addition, functional units in the embodiments of the present disclosure may be integrated into one processing unit, or each of the units may exist alone physically, or two or more units are integrated into one unit.

The foregoing descriptions are merely non-limiting examples of specific implementations and are not intended to limit the protection scope, which is intended to cover any variation or replacement readily determined by a person of ordinary skill in the art. Therefore, the claims shall define the protection scope.

1. A health analysis method for blood glucose management, comprising:
obtaining a plurality of blood glucose data of a user during a detection time period, wherein the plurality of blood glucose data comprise a current blood glucose value detected at a current moment;
determining a time in range ratio of the user according to the plurality of blood glucose data and a glucose standard range;
predicting a time when a blood glucose value of the user meets the glucose standard range and a time when the time in range ratio meets a qualified condition according to the current blood glucose value and the time in range ratio; and
analyzing predicted results to provide a blood glucose prediction curve to the user.

2. The health analysis method according to claim 1, wherein the health analysis method further comprises:
analyzing the predicted results to provide a blood glucose management path to the user.

3. The health analysis method according to claim 1, wherein the health analysis method further comprises:
analyzing the predicted results to provide a targeted target to the user, wherein the targeted target comprises a predicted target blood glucose value and a time for reaching the predicted target blood glucose value.

4. The health analysis method according to claim 1, wherein determining the time in range ratio of the user according to the plurality of blood glucose data and the glucose standard range comprises:
determining a number of standard detections during the detection time period, wherein one or more blood glucose values in the blood glucose data obtained by performing the standard detections are within the glucose standard range; and
determining the time in range ratio according to a ratio of the number of standard detections to a total number of detections during the detection time period.

5. The health analysis method according to claim 4, wherein predicting the time when the blood glucose value of the user meets the glucose standard range and the time when the time in range ratio meets the qualified condition according to the current blood glucose value and the time in range ratio comprises:
in response to the current blood glucose value being higher than the glucose standard range, predicting a first predicted time when the blood glucose value of the user meets the glucose standard range according to the current blood glucose value and a blood glucose decreasing speed; and
predicting a second predicted time when the time in range ratio meets the qualified condition according to the first predicted time and the time in range ratio.

6. The health analysis method according to claim 5, wherein the health analysis method further comprises:
obtaining motion data of the user in real time; and
determining the blood glucose decreasing speed according to the motion data.

7. The health analysis method according to claim 5, wherein predicting the second predicted time when the time in range ratio meets the qualified condition according to the first predicted time and the time in range ratio comprises:
determining a number of first predicted detections required to be experienced in that the blood glucose value of the user meets the glucose standard range according to the first predicted time;
determining a number of second predicted detections required to be experienced in predicting that the time in range ratio meets the qualified condition according to the number of first predicted detections, the number of standard detections and the total number of detections; and
determining the second predicted time according to the number of second predicted detections.

8. The health analysis method according to claim 7, wherein analyzing the predicted results to provide the blood glucose prediction curve to the user comprises:
determining a predicted target blood glucose value; and
drawing the blood glucose prediction curve according to the predicted target blood glucose value, the current blood glucose value, the first predicted time, the second predicted time and the blood glucose decreasing speed.

9. The health analysis method according to claim 8, wherein determining the predicted target blood glucose value comprises:
calculating a blood glucose estimated value according to the current blood glucose value, the first predicted time, the second predicted time and the blood glucose decreasing speed;
in response to the blood glucose estimated value being greater than or equal to a first preset value, determining the blood glucose estimated value as the predicted target blood glucose value; and
in response to the blood glucose estimated value being less than the first preset value, determining the first preset value as the predicted target blood glucose value.

10. The health analysis method according to claim 8, wherein analyzing the predicted results to provide the targeted target to the user comprises:
determining the targeted target according to the predicted target blood glucose value, the first predicted time and the second predicted time.

11. The health analysis method according to claim 8, wherein analyzing the predicted results to provide the blood glucose management path to the user comprises:
determining a predicted motion time of the user according to the current blood glucose value, the blood glucose decreasing speed and the predicted target blood glucose value; and
providing the blood glucose management path according to the predicted motion time.

12. The health analysis method according to claim 11, wherein providing the blood glucose management path according to the predicted motion time comprises:
providing the blood glucose management path according to the predicted motion time and a current time; or
providing the blood glucose management path according to the predicted motion time and user personal data.

13. The health analysis method according to claim 4, wherein predicting the time when the blood glucose value of the user meets the glucose standard range and the time when the time in range ratio meets the qualified condition according to the current blood glucose value and the time in range ratio comprises:
in response to the current blood glucose value being within the glucose standard range, predicting a second predicted time when the time in range ratio meets the qualified condition according to the time in range ratio.

14. The health analysis method according to claim 4, wherein predicting the time when the blood glucose value of the user meets the glucose standard range and the time when the time in range ratio meets the qualified condition according to the current blood glucose value and the time in range ratio comprises:
in response to the current blood glucose value being lower than the glucose standard range, predicting a first predicted time when the blood glucose value of the user meets the glucose standard, range according to the current blood glucose value and a blood glucose rising speed; and
predicting a second predicted time when the time in range ratio meets the qualified condition according to the first predicted time and the time in range ratio.

15. The health analysis method according to claim 14, wherein predicting the second predicted time when the time in range ratio meets the qualified condition according to the first predicted time and the time in range ratio comprises:
determining a number of first predicted detections required to be experienced in that the blood glucose value of the user meets the glucose standard range according to the first predicted time;
determining a number of second predicted detections required to be experienced in predicting that the time in range ratio meets the qualified condition according to the number of first predicted detections, the number of standard detections and the total number of detections; and
determining the second predicted time according to the number of second predicted detections.

16. The health analysis method according to claim 15, wherein analyzing the predicted results to provide the blood glucose prediction curve to the user comprises:
determining a predicted target blood glucose value; and
drawing the blood glucose prediction curve according to the predicted target blood glucose value, the current blood glucose value, the first predicted time, the second predicted time and the blood glucose rising speed.

17. The health analysis method according to claim 16, wherein determining the predicted target blood glucose value comprises:
calculating a blood glucose estimated value according to the current blood glucose value, the first predicted time, the second predicted time and the blood glucose rising speed;
in response to the blood glucose estimated value being less than or equal to a second preset value, determining the blood glucose estimated value as the predicted target blood glucose value; and
in response to the blood glucose estimated value being greater than the second preset value, determining the second preset value as the predicted target blood glucose value.

18. The health analysis method according to claim 1, wherein when the time in range ratio meets the qualified condition comprises: the time in range ratio is greater than or equal to 70%.

19. (canceled)

20. An electronic device, comprising a processor and a memory,
the memory storing a computer program, the computer program being executed by the processor to implement the health analysis method for blood glucose management according to claim 1.

21. A non-volatile computer-readable storage medium comprising a computer program, wherein the computer program, when executed by one or more processors, causes the processors to implement the health analysis method for blood glucose management according to claim 1.
